(19) United States
(12) Patent Application Publication
(10) Pub. No.: US 2005/0187388 A1
(43) Pub. Date: Aug. 25, 2005

(54) Method for the preparation of aryl ethers

(76) Inventors: Mateusz Cebula, Portage, MI (US); Kevin Edward Henegar, Ann Arbor, MI (US)

Correspondence Address: WARNER-LAMBERT COMPANY, 2800 PLYMOUTH RD, ANN ARBOR, MI, 48105, US

(21) Appl. No.: 11/059,895

(22) Filed: Feb. 17, 2005

Related U.S. Application Data
(60) Provisional application No. 60/546,486, filed on Feb. 20, 2004.

(51) Int. Cl. C07D 265/32

(52) U.S. Cl. 544/174

(57) ABSTRACT

The invention provides a method of preparing a compound of formula (I): 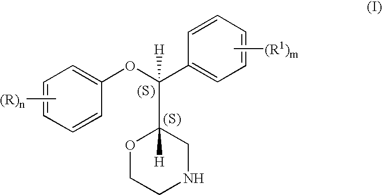
wherein R, R¹, n and m are as defined herein, or a pharmaceutically acceptable salt thereof.

This application is a United States utility application, which claims the benefit of priority to U.S. Provisional Application No. 60/546,486, filed Feb. 20, 2004.

### FIELD OF THE INVENTION

The present invention relates to an improved and enantioselective method for preparing certain aryl ethers, including (S,S)-reboxetine.

### BACKGROUND OF THE INVENTION

U.S. Pat. No. 4,229,449 discloses compounds of formula (A) 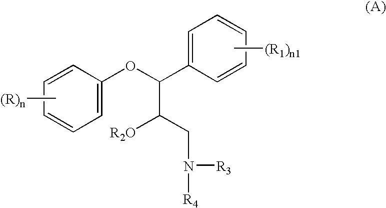
wherein

n and n1 are, independently, 1, 2 or 3;

each or the groups R and R₁, which may be the same or different, is hydrogen; halogen; halo-C₁-C₆alkyl; hydroxy; C₁-C₆alkoxy; C₁-C₆alkyl optionally substituted; aryl-C₁-C₆alkyl optionally substituted; aryl-C₁-C₆alkoxy optionally substituted; —NO₂; NR₅R₆ wherein R₅ and R₆ are, independently, hydrogen or C₁-C₆ alkyl, or two adjacent R groups or two adjacent R₁ groups, taken together, form a —O— CH₂—O— radical;

R₂ is hydrogen; C₁-C₁₂ alkyl optionally substituted, or aryl-C₁-C₆ alkyl;

each of the groups R₃ and R₄, which may be identical or different, is hydrogen, C₁-C₆alkyl optionally substituted, C₂-C₄alkenyl, C₂-C₄alkynyl, aryl-C₁-C₄ alkyl optionally substituted, C₃-C₇ cycloalkyl optionally substituted, or R₃ and R₄ with the nitrogen atom to which they are bounded form a pentatomic or hexatomic saturated or unsaturated, optionally substituted, heteromonocyclic radical optionally containing other heteroatoms belonging to the class of O, S and N;

or R₂ and R₄, taken together, form a —CH₂—CH₂— radical;

or a pharmaceutically acceptable salt thereof.

The compounds are disclosed to possess antidepressant activity.

In particular, U.S. Pat. No. 4,229,449 discloses the compound:

2-[α-(2-ethoxyphenoxy)benzyl]morpholine: 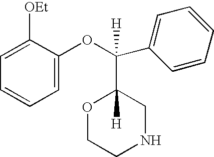
and pharmaceutically acceptable salts thereof, which possess useful antidepressant properties. This compound is also known as reboxetine.

Reboxetine does not act like most antidepressants. Unlike tricyclic antidepressants, and even selective serotonin reuptake inhibitors (SSRIs), reboxetine is ineffective in the 8-OH-DPAT hypothermia test, indicating that reboxetine is not a SSRI. See Brian E. Leonard, “Noradrenaline in basic models of depression.” European-Neuropsychopharmacol, 7 Suppl. 1 pp. S 11-6 and S71-3 (April 1997), incorporated herein in its entirety by reference thereto. Reboxetine is a selective norepinephrine reuptake inhibitor, with only marginal serotonin and no dopamine reuptake inhibitory activity. Reboxetine displays no anticholinergic binding activity in different animal models, and is substantially devoid of monoamine oxidase (MAO) inhibitory activity.

Many organic compounds exist in optically active forms, i.e., they have the ability to rotate the plane of plane-polarized light. In describing an optically active compound the prefixes R and S are used to denote the absolute configuration of the molecule about its chiral center(s). The prefixes D and L, or (+) or (−), designate the sign of rotation of plane-polarized light by the compound, with L or (−) meaning that the compound is levorotatory. In contrast, a compound prefixed with D or (+) is dextrorotatory. There is no correlation between nomenclature for the absolute stereochemistry and for the rotation of an enantiomer. Thus, D-lactic acid is the same as (−)-lactic acid, and L-lactic acid is the same as (+)-lactic acid. For a given chemical structure, each of a pair of enantiomers are identical except that they are non-superimposable mirror images of one another. A specific stereoisomer may also be referred to as an enantiomer, and a mixture of such isomers is often called an enantiomeric, or racemic, mixture.

When two chiral centers exist in one molecule, there are four possible stereoisomers: (R,R), (S,S), (R,S), and (S,R). Of these, (R,R) and (S,S) are an example of a pair of enantiomers (mirror images of each other), which typically share chemical properties and melting points just like any other enantiomeric pair. The mirror images of (R,R) and (S,S) are not, however, superimposable on (R,S) and (S,R). This relationship is called diastereoisomeric, and the (S,S) molecule is a diastereoisomer of the (R,S) molecule, whereas the (R,R) molecule is a diastereoisomer of the (S,R) molecule.

Chemically, reboxetine has two chiral centers and, therefore, exists as two enantiomeric pairs of diastereomers, the (R,R) and (S,S) enantiomeric pair and the (R,S) and (S,R) enantiomeric pair. Currently, reboxetine is commercially available only as a racemic mixture of enantiomers, (R,R) and (S,S) in a 1:1 ratio, and reference herein to the generic name “reboxetine” refers to this enantiomeric, or racemic, mixture. Reboxetine is commercially sold under the trade names of EDRONAX™, PROLIFT™, VESTRA™, and NOREBOX™.

It is now known (see WO 01/01973, incorporated in its entirety by reference) that the (S,S)-enantiomer of reboxetine possesses greatly enhanced selectivity for norepinephrine reuptake over serotonin reuptake. Accordingly, WO 01/01973 discloses a method of selectively inhibiting reuptake of norepinephrine, the method comprising the step of administering a therapeutically effective amount of a composition to an individual, the composition comprising a compound having a pharmacological selectivity of serotonin (Kᵢ)/norepinephrine (Kᵢ) of at least about 5000. The document further discloses a number of novel uses of (S,S)-reboxetine, including use of (S,S)-reboxetine in the treatment of chronic pain, peripheral neuropathy, incontinence (including stress incontinence, genuine stress incontinence, and mixed incontinence), fibromyalgia and other somatoform disorders, and migraine headaches.

WO 00/39072 provides a method for the preparation of aryl ethers, including racemic reboxetine, from an intermediate amine, comprising:

h) reacting a compound of formula VIIa: 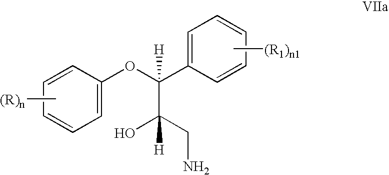
with a carboxylic acid of formula HOOCCH₂L or a reactive derivative thereof, wherein L is a leaving group, to give an amide of formula VIIIa:

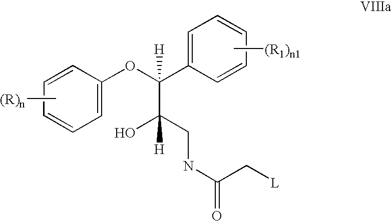

i) reacting the compound of formula VIIIa to give a compound of formula IXa: 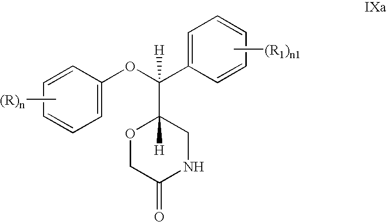
and

j) reducing the compound of formula IXa to give a corresponding compound of the following formula:

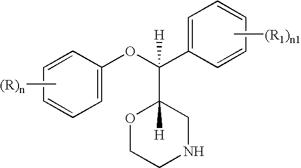

In the above formulae, R, R₁, n and n1 are as defined in U.S. Pat. No. 4,229,449 referred to above.

The above publications all disclose methods for producing reboxetine and related compounds in the form of a racemic mixture of the (R,R)- and (S,S)-enantiomers. A further resolution step is required in order to isolate the more potent (S,S)-enantiomer.

An alternative synthesis of (S,S)-reboxetine is described in GB-A-2167407. This document discloses a chiral synthesis of (S,S)-reboxetine starting from chiral phenylglycidic acid. However, no adequate chiral syntheses of the phenylglycidic acid exist so the chiral acid must be prepared by resolution, which is inefficient. The subsequent reduction to phenylglycidol is low yielding. Following this step, the synthesis described in GB-A-2167407 parallels the racemic synthesis: as remarked above, this synthesis exhibits poor selectivity and is low yielding and inefficient.

It would be desirable to provide an enantioselective synthesis of (S,S)-aryl ethers such as (S,S)-reboxetine and intermediates thereof which avoids the production of the unwanted (R,R)-enantiomer and allows the (S,S)-aryl ethers to be produced more efficiently and in a greater yield and purity than is allowed by the syntheses of the prior art.

We have surprisingly found that a novel synthesis according to the present invention allows (S,S)-aryl ethers such as (S,S)-reboxetine be produced enantioselectively, efficiently and in high yield and purity.

### SUMMARY OF THE INVENTION

The invention provides a method of preparing a compound of formula (I): 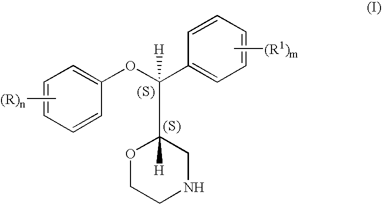
wherein:

n and m are, independently, 0 or an integer from 1 to 5; and

each of the groups R and R¹, which may be the same or different, is halogen; halo-C₁-C₆alkyl; hydroxy; C₁-C₆alkoxy; C₁-C₆alkyl optionally substituted by one or more substituents selected from halogen, hydroxy, C₁-C₆alkoxy, NR⁵R⁶ wherein R⁵ and R⁶ are, independently, hydrogen or C₁-C₆ alkyl, or —CONR⁵R⁶ wherein R⁵ and R⁶ are, independently, hydrogen or C₁-C₆ alkyl); aryl-C₁-C₆alkyl wherein the aryl ring is optionally substituted by one or more substituents selected from C₁-C₆ alkyl, halogen, halo-C₁-C₆alkyl, hydroxy, C₁-C₆alkoxy, and NR⁵R⁶ wherein R⁵ and R⁶ are, independently, hydrogen or C₁-C₆ alkyl; aryl-C₁-C₆alkoxy wherein the aryl ring is optionally substituted by one or more substituents selected from C₁-C₆alkyl, halogen, halo-C₁-C₆alkyl, hydroxy, C₁-C₆alkoxy, and NR⁵R⁶ wherein R⁵ and R⁶ are, independently, hydrogen or C₁-C₆ alkyl; -NO₂; NR⁵R⁶ wherein R⁵ and R⁶ are, independently, hydrogen or C₁-C₆ alkyl, or two adjacent R groups or two adjacent R¹ groups, taken together, form a —O—CH₂—O— radical;

or a pharmaceutically acceptable salt thereof;

comprising the steps:

(a) reaction of a compound of formula (II): 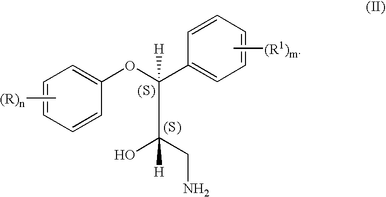
wherein R, R¹, n and m are as defined above, with a compound of formula X—CH₂—C(═O)—X′, wherein X and X′ are independently a leaving group or a group convertible in situ to a leaving group, in a non-polar organic solvent, optionally in the presence of water, to give a compound of formula (III):

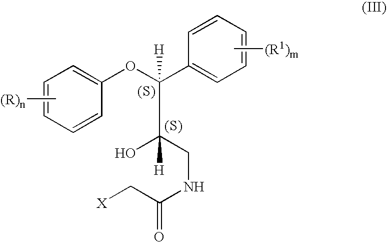

(b) reaction of the compound of formula (III) with a base in a non-polar organic solvent, to give a compound of formula (IV): 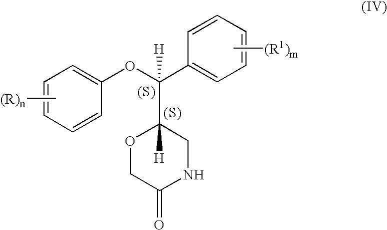
wherein R, R¹, n and m are as defined above, and

(c) reduction of the compound of formula (IV) with a reducing agent in a non-polar organic solvent to give a compound of formula (I); and optionally

(d) forming a pharmaceutically acceptable salt of the compound of formula (I);

wherein steps (a), (b) and (c) are carried out sequentially and in the same non-polar organic solvent.

Carrying out steps (a), (b) and (c) in the same non-polar organic solvent confers significant advantages over the prior art in that solvent changes are dispensed with. This allows the synthesis to proceed more efficiently and in greater yields than those of the prior art.

We have surprisingly found that by carrying out process steps (a), (b) and (c) sequentially and in the same non-polar organic solvent, the reaction proceeds efficiently and in good yield. This is contrary to what would be expected as the process steps, in particular step (b), would not be expected to proceed successfully in a non-polar organic solvent.

### DETAILED DESCRIPTION OF PREFERRED EMBODIMENTS

In the compounds of formulae (I) through (IV), each of the groups R and R¹, which may be the same or different, is halogen; halo-C₁-C₆alkyl; hydroxy; C₁-C₆alkoxy; C₁-C₆ alkyl optionally substituted by one or more substituents selected from halogen, hydroxy, C₁-C₆alkoxy, NR⁵R⁶ wherein R⁵ and R⁶ are, independently, hydrogen or C₁-C₆ alkyl, or —CONR⁵R⁶ wherein R⁵ and R⁶ are, independently, hydrogen or C₁-C₆ alkyl; aryl-C₁-C₆alkyl wherein the aryl ring is optionally substituted by one or more substituents selected from C₁-C₆alkyl, halogen, halo-C₁-C₆alkyl, hydroxy, C₁-C₆ alkoxy, and NR⁵R⁶ wherein R⁵ and R⁶ are, independently, hydrogen or C₁-C₆ alkyl; aryl-C₁-C₆ alkoxy wherein the aryl ring is optionally substituted by one or more substituents selected from C₁-C₆alkyl, halogen, halo-C₁-C₆ alkyl, hydroxy, C₁-C₆ alkoxy, and NR⁵R⁶ wherein R⁵ and R⁶ are, independently, hydrogen or C₁-C₆ alkyl; —NO₂; NR⁵R⁶ wherein R⁵ and R⁶ are, independently, hydrogen or C₁-C₆ alkyl, or two adjacent R groups or two adjacent R¹ groups, taken together, form a —O—CH₂—O— radical; and n and m are, independently, 0 or an integer from 1 to 5.

The term “alkyl” means a straight or branched chain saturated hydrocarbon group containing 1 to 6 carbon atoms.

The term “haloalkyl” means an alkyl group, as defined above, which is substituted by one or more halogen atoms.

The term “alkoxy” means “alkyl-O—”, wherein the alkyl group is as defined above.

The term “aryl” means a phenyl or naphthyl group.

Suitably, the groups R and R¹, which may be the same or different, are selected from hydroxy and C₁-C₆alkoxy. Preferably, R is methoxy or ethoxy, more preferably ethoxy.

Preferably, n is 1 or 2, more preferably 1.

Preferably, m is 0 or 1, more preferably 0.

Step (a)

The process of the present invention begins with reaction of a compound of formula (II) with a compound of formula X—CH₂—C(═O)—X′ in a non-polar organic solvent to give a compound of formula (III).

In the compound of formula X—CH₂—C(═O)—X′, the groups X and X′ are each independently a leaving group or a group convertible in situ to a leaving group. The precise nature of the groups X and X′ are not critical provided they are usually capable of acting as leaving groups or are capable of being converted in situ to leaving groups. Examples of suitable leaving groups include halogen atoms and sulfonyloxy groups (for example, methanesulfonyloxy, benzenesulfonyloxy or p-toluenesulfonyloxy). Examples of suitable groups capable of being converted in situ to leaving groups include hydroxy groups. These groups may be converted in situ to a leaving group in a conventional manner. For example, a hydroxy group may be converted to a p-toluenesulfonyloxy group by reaction with p-toluenesulfonyl chloride in pyridine.

Preferably X and X′ are halogen atoms, more preferably chlorine or bromine atoms.

The reaction is preferably carried out in the presence of a base. The nature of the base is not especially critical provided that it is capable of acting as a base. Examples of suitable bases include alkali metal carbonates such as lithium carbonate, sodium carbonate, potassium carbonate, rubidium carbonate and caesium carbonate, and organic amines such as trimethylamine, triethylamine, pyridine, methyidiethylamine, dimethylethylamine, tri n-propylamine, triisopropylamine, diisopropylethylamine, tributylamines, and higher trialkylamines, picolines, lutidines, and collidines, 2-methyl-5-ethylpyridine (lonza pyridine), 2,6-di-t-butylpyridine, 2,6-di-t-butyl-4-methylpyridine and alkyl quinolines and isoquinolines, N-methylpiperidine, N-methylpyrrolidine, N-methylmorpholine, and higher alkyl analogues of these compounds. Alkali metal carbonates are preferred and sodium carbonate is especially preferred.

The reaction is carried out in the presence of a non-polar organic solvent, the nature of which is not especially critical provided it is inert to the reaction, is non-polar and is capable of dissolving the reactants at least to some extent. Examples of suitable solvents include aliphatic hydrocarbons such as pentanes, hexanes, heptanes and octanes, and aromatic hydrocarbons such as benzene, toluene, xylenes, ethylbenzene, mesitylenes and tetralin. When the base is an inorganic base such as an alkali metal carbonate, water may also preferably be present. It is preferred that the solvent is a biphasic mixture of an aromatic hydrocarbon, particularly toluene, and water.

The reaction temperature depends on various factors such as the nature of the reagent and the solvent. However, it is typically from −20° C. to room temperature, and preferably 0° C. to 10° C.

The reaction time depends on various factors such as the nature of the reagents, the solvent and the temperature. However, it is typically from 10 minutes to 6 hours, and preferably from 30 minutes to 3 hours.

After completion of the reaction, a solution containing the compound of formula (III) is worked up in a conventional manner. For example, the solution of the compound of formula (III) in the non-polar organic solvent may be washed with an aqueous solution such as water or sodium chloride in order to remove any ionic species present, filtered to remove any solid matter, and dried by distillation, preferably under reduced pressure. However, the compound of formula (III) is not isolated from the solution. Rather, the solution containing compound of formula (III) is reacted directly with a base in the same non-polar organic solvent in the next step.

Step (b)

In this step the compound of formula (III) is cyclised by reaction with a base in the same non-polar organic solvent used in step (a) to give a compound of formula (IV).

The nature of the base is not especially critical provided that it is capable of acting as a base and is at least partially soluble in a non-polar organic solvent. Examples of suitable bases include alkali metal alkoxides such as sodium t-amylate, lithium t-butoxide, and potassium t-butoxide, and alkali metal bis(trimethylsilyl)amides (alkali metal hexamethyldisilazides) such as lithium hexamethyldisilazide, sodium hexamethyldisilazide and potassium hexamethyldisilazide. Alkali metal alkoxides are preferred and sodium t-amylate is especially preferred.

The reaction is carried out in the presence of the same non-polar organic solvent as used in step (a). Examples of suitable solvents include those listed above in relation to step (a). It is preferred that the solvent is an aromatic hydrocarbon, particularly toluene.

The reaction temperature depends on various factors such as the nature of the reagent and the solvent. However, it is typically from −50° C. to room temperature, and preferably −20° C. to 10° C.

The reaction time depends on various factors such as the nature of the reagents, the solvent and the temperature. However, it is typically from 10 min to 12 hours, and preferably from 30 min to 2 hours.

After completion of the reaction, a solution containing the compound of formula (IV) is worked up in a conventional manner. For example, the solution of the compound of formula (IV) in the non-polar organic solvent may be washed with an aqueous solution such as water or sodium chloride in order to remove any ionic species present, filtered to remove any solid matter, and dried by distillation, preferably under reduced pressure. However, the compound of formula (IV) is not isolated from the solution. Rather, the solution containing the compound of formula (IV) is reacted directly with a reducing agent in the same non-polar organic solvent in the next step.

Step (c)

In this step the compound of formula (IV) is reduced with a reducing agent in the same non-polar organic solvent used in steps (a) and (b) to give a compound of formula (I).

The reducing agent used is not particularly critical provided it is capable of reducing an amide to the corresponding amine and is at least partially soluble in a non-polar organic solvent. Examples of suitable reducing agents include lithium aluminium hydride, sodium bis(2-methoxyethoxy)aluminium hydride (Vitride®) and borane, of which sodium bis(2-methoxyethoxy)aluminium hydride (Vitride®) is preferred.

The reaction is carried out in the presence of the same non-polar organic solvent as used in steps (a) and (b). Examples of suitable solvents include those listed above in relation to step (a). It is preferred that the solvent is an aromatic hydrocarbon, particularly toluene.

The reaction temperature depends on various factors such as the nature of the reagent and the solvent. However, it is typically from −20° C. to room temperature, and preferably −10° C. to 10° C.

The reaction time depends on various factors such as the nature of the reagents, the solvent and the temperature. However, it is typically from 30 minutes to 10 hours, and preferably from 1 to 4 hours.

After completion of the reaction, the compound of formula (I) is isolated from the reaction mixture by a conventional method. For example, the compound may be extracted into water as an acid addition salt by the addition of an acid such as hydrochloric acid, neutralised by the addition of a base such as sodium hydroxide, and then extracted using an organic solvent. The solution may be washed with an aqueous solution such as aqueous sodium carbonate solution, and then optionally concentrated to remove the solvent. The product may further be purified by conventional methods such as column chromatography. Alternatively, the product may be reacted directly with an acid to produce a pharmacologically acceptable salt of the compound of formula (I) in the next step.

Step (d)

In this optional step, the compound of formula (I) is converted to a pharmacologically acceptable salt by reaction with an acid.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Preferred examples of salts include the mesylate, fumarate and succinate salts, and the succinate is especially preferred.

For a review on suitable salts, see “Handbook of Pharmaceutical Salts: Properties, Selection, and Use” by Stahl and Wermuth (Wiley-VCH, Weinheim, Germany, 2002).

A pharmaceutically acceptable salt of a compound of formula (I) may be readily prepared in a conventional manner by mixing together solutions of the compound of formula (I) and the desired acid, as appropriate. The salt may precipitate from solution and be collected by filtration or may be recovered by evaporation of the solvent. The degree of ionisation in the salt may vary from completely ionised to almost non-ionised.

Compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 wt % to 5 wt % of the tablet, and glidants may comprise from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 wt % to 10 wt%, preferably from 0.5 wt % to 3 wt % of the tablet.

Other possible ingredients include anti-oxidants, colourants, flavouring agents, preservatives and taste-masking agents.

Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Verma etal, Pharmaceutical Technology On-line, 25(2), 1-14 (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and PGLA microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958 by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or HPMC), blisters and cartridges for use in an inhaler or insufflator may be formulated to contain a powder mix of the compound of the invention, a suitable powder base such as lactose or starch and a performance modifier such as /-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid) (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff”.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

### EXAMPLES

A preferred embodiment of the present invention will now be described in more detail with reference to the following Examples. These are intended to illustrate and not limit the scope of the invention.

### Example 1

### (2S,3S)-N-Chloroacetyl-3-(2-ethoxyphenoxy)-2-hydroxy-3-phenylpropylamine

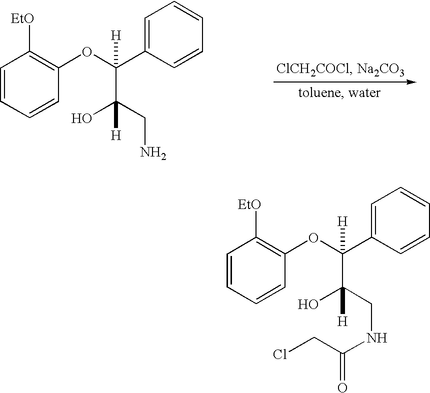

(2S,3S)-3-(2-Ethoxyphenoxy)-2-hydroxy-3-phenylpropylamine (20 g) was stirred with toluene (200 mL) to form a slurry. A solution of sodium carbonate (14.76 g) in water (74 mL) was added and the two-phase mixture was stirred slowly and cooled to 5° C. A solution of chloroacetyl chloride (13.36 g) in toluene (66 mL) was added dropwise over 1 h. When the reaction was complete, water (200 mL) was added and the mixture heated to 50° C. The phases were separated and the aqueous phase was discarded. The organic phase was washed with water (200 mL) and then with saturated aqueous NaCI solution (200 mL). The toluene was distilled under reduced pressure until the water content, as measured by KF assay, was less than 0.20%. The total volume was adjusted to 150 mL by the addition of toluene to yield a solution of the title compound.

### Example 2

### (2S,3S)-2-[α-(2-Ethoxyphenoxy)benzyl]morpholine-5-one

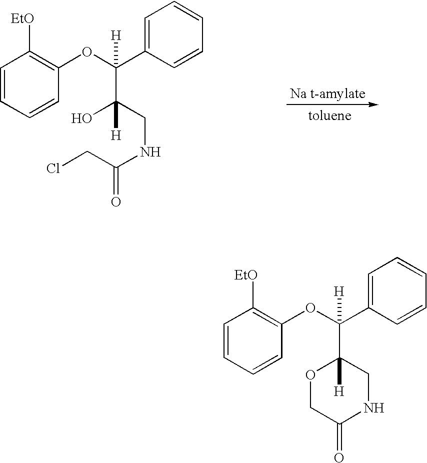

A solution of sodium t-amylate (14.97 g) in toluene (247.3 mL) was cooled to −5° C. The solution of (2S,3S)-N-chloroacetyl-3-(2-ethoxyphenoxy)-2-hydroxy-3-phenylpropylamine from Example 1 was added slowly maintaining the temperature of the reaction less than −5° C. When the reaction was complete, water (309 mL) was added and the mixture was stirred for 10 minutes. The phases were separated and the organic solution was washed with water (2×309 mL). The organic layer was filtered through a plug of magnesol, and the magnesol was washed with toluene (50 mL). The toluene was distilled under reduced pressure until the water content, as measured by KF assay, was less than 0.05%. The total volume was adjusted to 118 mL by the addition of toluene to give a solution of the title compound.

### Example 3

### (2S,3S)-2-[α-(2-ethoxyphenoxy)benzyl]morpholine

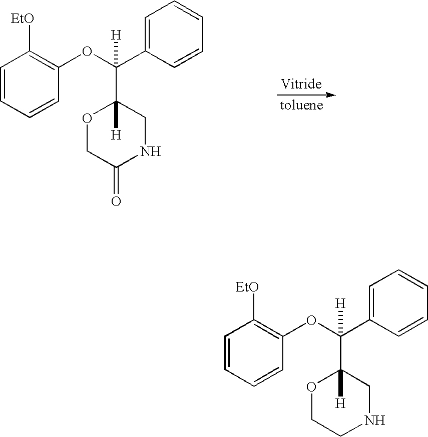

Toluene (100 mL) and Vitride® (65 wt % solution in toluene, 102.2 mL) were stirred and cooled to 0 to −10° C. The solution of (2S,3S)-2-[α-(2-ethoxyphenoxy)-benzyl]morpholine-5-one from Example 2 was added at a constant rate to the Vitride® solution keeping the temperature of the reaction mixture between 0 and +10° C. The mixture was stirred at 0 to 10° C. for about 1 h. The reaction was quenched by the addition of a solution of 50% NaOH (21 mL) dissolved in water (184 mL). The mixture was allowed to warm to 45-55° C. and then maintained at 45-55° C. by cooling. The phases were separated. The organic phase was washed with 5% (w/v) aqueous Na₂CO₃ solution (3×274 mL) and the aqueous phases were discarded. Water (335 mL) was added to the toluene solution. The mixture was stirred vigorously and 8% HCl solution was added until the pH of the aqueous phase was 3.0-3.5. The phases were separated and the organic phase was discarded. Toluene (408 mL) was added to the aqueous phase and then 10% NaOH was added until the pH was 10-12. The phases were separated and the aqueous phase was discarded. The organic layer was washed with 5% (w/v) Na₂CO₃ solution (3×100 mL) and then once with water (100 mL). The organic phase was concentrated to an oil under reduced pressure and then isopropyl alcohol was added and distilled until the residual toluene level was less than 0.3% to yield a solution of (2S,3S)-2-[α-(2-ethoxyphenoxy)benzyl]morpholine.

### Example 4

### (2S,3S)-2-[α-(2-Ethoxyphenoxy)benzyl]morpholine succinate (2S, 3S)-Reboxetine Succinate

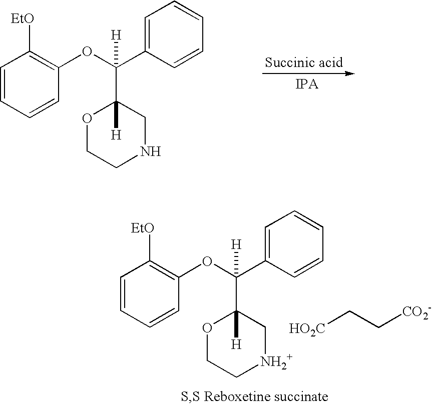

The solution of (2S,3S)-2-[α-(2-ethoxyphenoxy)benzyl]morpholine from Example 3 was dissolved in isopropyl alcohol (474.6 mL). Succinic acid (9.9 g) was added to the solution and the mixture was heated to 75° C. and stirred until all the solids had dissolved. The solution was stirred and cooled slowly to 20-25° C. The mixture was stirred at 20-25° C. for 2 hours and the crystals were filtered, washed with isopropyl alcohol (88 mL), and dried on a nitrogen press. Yield: 30.0 g of the title compound. This material assayed at greater than 99% ee.

Chiral HPLC assay:

| | |
|---|---|
| Column: | Chiracel OJ-H |
| Mobile phase | 25:75 hexanes:isopropanol + 0.5% diethylamine |
| Flow | 0.75 mL/minute |
| Detector | 275 nm |
| Run time | 30 minutes |

Retention times

(2R,3R)-2-[α-(2-Ethoxyphenoxy)benzyl]morpholine 5.6 minutes

(2S,3S)-2-[α-(2-Ethoxyphenoxy)benzyl]morpholine 9.0 minutes

mp 145.4-151.9° C.

[α]²⁰_{D} (C=10)+14.64°

¹H NMR (400.13 MHz, d₆-DMSO) δ 1.33 (t, J=7.1 Hz, 3H), 2.34 (s, 4H), 2.61-2.82 (m, 3H), 3.50 (m, 2H), 3.83 (m, 2H), 4.01 (q, J=7.1 Hz, 2H), 5.27 (d, J=6.0 Hz, 1H), 6.66-6.96 (m, 4H), 7.22-7.54 (m, 5H).

¹³C NMR (100.62 MHz, d₆-DMSO) δ 14.88, 30.19, 39.10, 39.31, 39.51, 39.72, 44.14 45.77, 64.10, 65.89, 77.46, 80.80, 114.42, 116.68, 120.73, 127.27, 128.20, 137.76, 147.34, 149.04, 174.38.

1. A method of preparing a compound of formula (I): 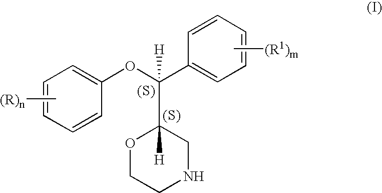
wherein:
n and m are, independently, 0 or an integer from 1 to 5; and each of the groups R and R¹, which may be the same or different, is halogen; halo-C₁-C₆ alkyl; hydroxy; C₁-C₆ alkoxy; C₁-C₆ alkyl optionally substituted by one or more substituents selected from halogen, hydroxy, C₁-C₆alkoxy, NR⁵R⁶ wherein R⁵ and R⁶ are, independently, hydrogen or C₁-C₆ alkyl, and —CONR⁵R⁶ wherein R⁵ and R⁶ are, independently, hydrogen or C₁-C₆ alkyl; aryl-C₁-C₆alkyl wherein the aryl ring is optionally substituted by one or more substituents selected from C₁-C₆ alkyl, halogen, halo-C₁-C₆alkyl, hydroxy, C₁-C₆alkoxy, and NR⁵R⁶ wherein R⁵ and R⁶ are, independently, hydrogen or C₁-C₆ alkyl; aryl-C₁-C₆alkoxy wherein the aryl ring is optionally substituted by one or more substituents selected from C₁-C₆ alkyl, halogen, halo-C₁-C₆alkyl, hydroxy, C₁-C₆alkoxy, and NR⁵R⁶ wherein R⁵ and R⁶ are, independently, hydrogen or C₁-C₆ alkyl; —NO₂; NR⁵R⁶ wherein R⁵ and R⁶ are, independently, hydrogen or C₁-C₆ alkyl, or two adjacent R groups or two adjacent R¹ groups, taken together, form a —O—CH₂—O— radical;
or a pharmaceutically acceptable salt thereof;
comprising the steps:
(a) reaction of a compound of formula (II): 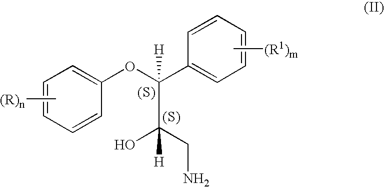
wherein R, R¹, n and m are as defined above, with a compound of formula X—CH₂—C(═O)—X′, wherein X and X′ are independently a leaving group or a group convertible in situ to a leaving group, in a non-polar organic solvent, optionally in the presence of water, to give a compound of formula (III): 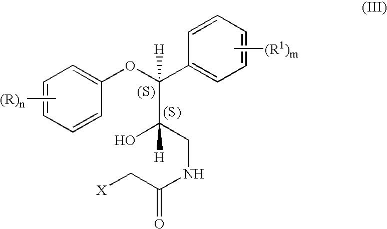
wherein R, R′, n and m are as defined above;
(b) reaction of the compound of formula (III) with a base in a non-polar organic solvent, to give a compound of formula (IV): 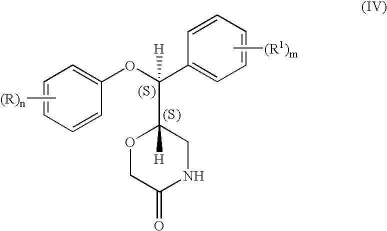
wherein R, R¹, n and m are as defined above, and
(c) reduction of the compound of formula (IV) with a reducing agent in a non-polar organic solvent to give a compound of formula (I); and optionally
(d) forming a pharmaceutically acceptable salt of the compound of formula (I);
wherein steps (a), (b) and (c) are carried out sequentially and in the same non-polar organic solvent.

2. A method according to claim 1, wherein steps (a), (b) and (c) are carried out in an aromatic hydrocarbon solvent.

3. A method according to claim 2, wherein steps (a), (b) and (c) are carried out in toluene solvent.

4. A method according to claim 1, wherein the groups R and R₁, which may be the same or different, are selected from hydroxy and C₁-C₆ alkoxy.

5. A method according to claim 4, wherein R is ethoxy.

6. A method according to claim 1, wherein n is 1.

7. A method according to claim 1, wherein m is 0.

8. A method according to claim 1, wherein the compound of formula (I) prepared is (2S,3S)-2-[α-(2-ethoxyphenoxy)benzyl]morpholine.

9. A method according to claim 1, wherein X and X′ are chlorine atoms.

10. A method according to claim 1, wherein step (a) is carried out in the presence of a base.

11. A method according to claim 10, wherein the base is sodium carbonate.

12. A method according to claim 1, wherein the base used in step (b) is an alkali metal alkoxide.

13. A method according to claim 12, wherein the base used in step (b) is sodium t-amylate.

14. A method according to claim 1, wherein the reducing agent used in step (c) is sodium bis(2-methoxyethoxy)aluminium hydride.

15. A method according to claim 1, wherein the pharmaceutically acceptable salt of the compound of formula (I) prepared in step (d) is the succinate.
